(19) United States
(12) Patent Application Publication
(10) Pub. No.: US 2009/0258408 A1
(43) Pub. Date: Oct. 15, 2009

(54) Method for the isolation of spiroplasma in mammals

(76) Inventor: Anthony Perry, Gilbert, AZ (US)

Correspondence Address: BIRCH STEWART KOLASCH & BIRCH, PO BOX 747, FALLS CHURCH, VA, 22040-0747, US

(21) Appl. No.: 11/919,975

(22) PCT Filed: May 8, 2006
(86) PCT No.: PCT/US06/17397

§ 371 Date: Nov. 6, 2007

Related U.S. Application Data
(60) Provisional application No. 60/678,228, filed on May 6, 2005.

(51) Int. Cl. C12N 1/20

(52) U.S. Cl. 435/252.1

(57) ABSTRACT

The present invention is directed to a method of isolating and culturing spiroplasma by obtaining a tissue sample from a bovine; inoculating embryonated eggs with the tissue sample; mixing fluid from the inoculated egg with culture media; culturing the media mixed with the egg fluid at approximately 32° C. for preferably greater than a month to establish a spiroplasma culture.

### FIELD OF THE INVENTION

The subject invention is directed towards a method for isolating spiroplasma and culturing spiroplasma from a mammal.

### BACKGROUND OF THE INVENTION

Transmissible spongiform encephalopathies (TSEs) are a family of diseases of humans and animals characterized by spongy degeneration of the brain with severe and fatal neurological signs and symptoms. TSEs include the following diseases:
In humans
   CJD Creutzfeldt-Jakob disease (and new variant CJD—nvCJD or vCJD)
   GSS Gerstmann Sträussler Scheinker syndrome
   FFI Fatal familial insomnia
   Kuru
   Alpers syndrome (hypothesized)
In other vertebrate animals
   Scrapie in sheep, goats and small ruminants
   Bovine spongiform encephalopathy (BSE) in cattle
   Chronic wasting disease (CWD) in cervids
   Transmissible mink encephalopathy (TME) in mink
   Feline spongiform encephalopathy (FSE) in cats
      BSE is a transmissible, neuro-degenerative fatal brain disease of cattle. The disease has a long incubation period of 4-5 years and it is fatal for cattle within weeks to months of its onset. BSE is of particular importance because of the transmission of the disease to humans through the consumption of beef obtained from a diseased animal. It has been estimated that BSE has had a total net cost to the UK economy of over £3.7 billion by the end of 2002, equivalent to 0.1-0.2% of the total national economy of the UK. (Ministry of Agriculture, Fisheries and Food, “The BSE Inquiry”, Vol. 10, (2000)). The World Organization for Animal Health, (Office International des Epizooties) provides an international standard for the diagnosis of bovine spongiform encephalopathy. See World Organization for Animal Health, OIE Manual of Diagnostic Tests and Vaccines for Terrestrial Animals, 5th ed., chapter 2.3.13 (2004). Immunohistochemistry testing is considered the gold standard for BSE diagnosis and OIE's “SAF” Western blot is used when a sample is not suitable for immunohistochemistry. The Canadian government follows the OIE-compliant immunohistochemistry and SAF Western blot BSE diagnosis protocols. Canadian Food Inspection Agency, Fact Sheet: Canada's Protocols for BSE Surveillance (August 2005). Similarly, in the U.S., OIE-compliant immunohistochemistry and SAF Western blot protocols are used to diagnose BSE. Animal and Plant Health Inspection Service, Factsheet: BSE Confirmatory Tests (June 2005).

Transmissible spongiform encephalopathies (TSEs) are widely accepted to be caused by the misfolded prion protein PrP^{res}. Evidence for this hypothesis is primarily based upon the importance of PrP in susceptibility to disease and association of PrP^{res} with TSE (Pruisner SB. (1982) Novel proteinaceous infectious particles cause scrapie. Science 216 (4542) 136-144). Despite this, there are many observations that are unexplained by an infectious PrP^{res} protein-only hypothesis. The amount of PrP has not been shown to correlate with infection and recombinant PrP is not infectious.

An alternate hypothesis is that spiroplasma may be the direct causative agent or that spiroplasma induce alternative folding of PrP and enucleation of the prion. (Bastion, Neuropathol. Exp. Neurol. Vo. 64, No. 10, 833-838 (2005)).

Spiroplasmas are small, motile, wall-less prokaryotic organisms. (Tully et al., “the Genus Spiroplasma, in THE PROKARYOTES, 2^{nd} ed. Springer-Verlag, Chapter 89 (1984)). Because spiroplasma lack a cell wall they have a pleiomorphic morphology, including: helices of varying lengths, coccoid, clumped and branching helical forms. Spiroplasma range in size from 50-200 nm in diameter and 3-12 μm in length. The helices are polar, with a blunt adhesion end and a tapered end. Under stress, a spore-like 30-40 nm coccoid form predominates that is impervious to extreme environmental insult.

The primary hosts of spiroplasmas are insects and plants, with an estimated prevalence of 70 percent of insects infected. (Tully et al., 1984) Thus far, confirmed spiroplasma-caused diseases in mammals are limited to cataracts and a spongiform neuropathy in rodents. (Clark, J. of Infec. Dis. Vol. 114, No. 5, 476-487 (1964); Kern S, Schroder J, Reischl U, Lorenz B. (1998) Endogenous infection with spiroplasma in a premature baby. 96^{th} DOG Annual Meeting p 97.)

Evidence for the involvement of spiroplasma with TSEs includes morphological identification of spiroplasma by transmission electron microscope in a Creutzfeldt-Jakob disease (CJD) brain biopsy, development of a spongiform encephalopathy model in vivo by the spiroplasma inoculation of neo-natal rodents, demonstration that scrapie antisera cross reacts with spiroplasma proteins, and the presence of spiroplasma ribosomal DNA in TSE tissue.

To date however, spiroplasma have not been successfully isolated and cultured from mammalian tissues. The inability to isolate and culture spiroplasma from the mammalian tissue of a diseased animal has precluded the investigation of Koch's postulates of correlation of etiologic agent to disease state:

1. The agent must be present in every case of the disease.

2. The agent must be isolated from the host with the disease and grown in pure culture.

3. The specific disease must be reproduced when a pure culture of the agent is inoculated into a healthy susceptible host.

4. The agent must be recoverable from the experimentally infected host.

The thus far unsuccessful isolation of spiroplasma in mammals has hitherto eluded researchers and prevented exploration of spiroplasma as a candidate for the etiologic agent of TSEs. The present invention presents the successful isolation of spiroplasma from a tissue sample, followed by the establishment of cultures generated from the isolated spiroplasma.

### SUMMARY OF THE INVENTION

The present invention is drawn to a method of isolating and culturing spiroplasma from a mammal by:

obtaining a tissue sample from a mammal;

inoculating embryonated eggs with the tissue sample;

mixing yolk from the inoculated egg with culture media;

culturing the media mixed with the egg yolk at approximately 32° C. for more than four weeks to establish a spiroplasma culture.

In an alternative embodiment, the present invention is directed to a method of isolating and culturing spiroplasma from a bovine by:

obtaining a tissue sample from a bovine;

inoculating embryonated eggs with the tissue sample;

mixing fluid from the inoculated egg with culture media;

culturing the media mixed with the fluid from the egg at approximately 32° C. to establish a spiroplasma culture.

### DETAILED DESCRIPTION OF THE INVENTION

The present invention is directed to a method of isolating spiroplasma from mammals and culturing the spiroplasma thus isolated.

The present invention first isolates spiroplasma from the tissue of an animal, which has been diagnosed with a TSE. The diagnosis of TSE can be done using any acceptable means, such as the international standard for the diagnosis of bovine spongiform encephalopathy developed by OIE.

The tissue sample may be obtained from any tissue-type of the animal, with neuronal tissue being preferred. However, other tissue sources, such as muscle or blood, may also be used.

The mammalian subject from which the tissue sample is obtained may be any mammal, but of particular importance is the isolation of spiroplasma from bovine or human subjects.

The tissue sample obtained from the animal is homogenized, if necessary, in a suitable culture media, using any conventional means. Preferred culture medias include M1D, SP4 or H-1 media. (Jones A L, Witcomb R F, Williamson D L, Coan M E. (1977) Phytopathology 47 738-746.), The media optionally may be supplemented with antibiotics, such as ampicillin and polymyxin B, amino acids, such as L-Arginine, and sphingomyelin (Hackett, J. Science 237 525-527 (1987). The samples are then centrifuged to remove debris. The resulting supernatant is further purified by passage through a filter.

The resulting filtrate stock is then serially diluted and used to inoculate embryonated eggs. The embryonated eggs are typically 7-10 days old when used for inoculation.

The eggs are then incubated at 32-37° C., preferably approximately 36° C. The incubation temperature may be varied to control the growth rate of the embryos. At approximately 6-10 days, preferably approximately 8 days, fluid from the egg is harvested and a stock prepared by preservation at −70° C.

From the inoculated egg fluid stock preparation, suspension cultures are initiated by preparations dilutions (e.g. 1:2 and 1:10 dilutions) of egg fluid stock and culture medium.

The cultures are incubated at 32° C., typically for more than one month, preferably more than 5 or 6 weeks, and monitored by acidification of the media using, for example, the pH indicator phenol red. The cultures can be further monitored for morphology using, for example, dark-field microscopy, and genetically using, for example, PCR.

Once the cultures have reached log-phase, the cultures are filtered through a millipore filter with a pore size of approximately 220 nm and plated onto solid medium (for example, the solid medium disclosed in Tully J G, Whitcomb R F, Clark H F, Williamson D L. (1977) Pathogenic spiroplasmas: cultivation and vertebrate pathogenicity of a new spiroplasma. Science 195 892-894.), and incubated anaerobically. Following incubation, single colonies are selected and transferred to fresh broth for expansion.

Once the spiroplasma have been isolated from the tissue sample they can be further characterized. For example, the morphology of the isolated spiroplasma can be initially analyzed by bright-field, fluorescent, dark-field and/or electron microscopy. Motility can be determined by visualization during microscopy and time-lapse imaging. Staining protocols have been developed for imaging of spiroplasma to allow the visualization of the organism. To analyze the size and morphology transmission electron microscopy of thin sections or negative stains is necessary for detailed visualization.

The isolated spiroplasma can also be characterized genetically using PCR amplification and sequencing. For example, the ribosomal genes may be sequenced and compared to other sequences, using, for example, BLAST analysis, to phylogenetically categorize the isolated spiroplasma.

### EXEMPLIFIED EMBODIMENTS OF THE INVENTION

### Isolation of Spiroplasma:

Tissue samples of 500 mg are isolated from three diseased animals and three healthy controls, with the disease having been previously demonstrated by the Canadian Food Inspection Agency according to their standards. (Fact Sheet: Canada's Protocols for BSE Surveillance (August 2005))

Samples of approximately 100 mg are homogenized with 500 μl of M1D culture medium (Jones 1977) (supplemented with 500 units/ml ampicillin, 500 units/ml polymyxin B,L-Arginine 1.9 mg/ml, and sphingomyelin 25 mg/L), in 1.5 ml plastic centrifuge tubes using disposable plastic pestles. Debris is clarified by centrifugation for 1 minute at 1000 g. The resulting supernatant is further purified by passage through a 450 nm filter. The resulting filtrate stock is used for inoculation of embryonated chicken eggs.

Dilutions of 1:10, 1:100 and 1:1000 in M1D medium are prepared and 100 μl is inoculated into the yolk sack of six 7-10 day embryonated eggs. The eggs are then incubated at 36° C. and candled daily for 8 days to determine viability. At 8 days, fluid from the egg is harvested and a stock prepared by preservation at −70° C. M1D suspension cultures are initiated by preparations of 1:2 and 1:10 dilutions of egg fluid and medium.

Cultures are incubated at 32° C. and monitored by acidification of media pH indicator phenol red, dark-field microscopy, and PCR.

Log-phase cultures are filtered through a 220 nm millipore filter and plated onto solid (Tully J G, Whitcomb R F, Clark H F, Williamson D L. (1977) Pathogenic spiroplasmas: cultivation and vertebrate pathogenicity of a new spiroplasma. Science 195 892-894.) medium and incubated anaerobically. Following incubation, single colonies are selected and transferred to fresh broth for expansion.

### Characterization for Validation of Isolate.

Analysis of general biologic characteristics. The morphology of the isolated spiroplasma can be initially analyzed by bright-field, fluorescent, dark-field and/or electron microscopy. Motility can be determined by visualization during microscopy and time-lapse imaging. Staining protocols have been developed for imaging of spiroplasma to allow the visualization of the organism. To analyze the size and morphology transmission electron microscopy of thin sections or negative stains is necessary for detailed visualization.

Amplification and sequencing can be used to evaluate the presence of spiroplasma DNA. Samples of white blood cells from BSE and control animals are obtained by density gradient centrifugation of whole blood. Samples from cultures are pelleted by centrifugation and washed. DNA is extracted by guanidine thiocyanate and purified by phenol chloroform extraction. Sequences can be analyzed by Basic Local Alignment Search Tool (BLAST) and phylogenetically categorized based on homology to known spiroplasma sequences.

1. A method of isolating and culturing spiroplasma from a mammal which comprises: obtaining a tissue sample from a mammal; inoculating embryonated eggs with the tissue sample; mixing fluid from the inoculated egg with culture media; culturing the media mixed with the egg fluid at approximately 32° C. for more than four weeks to establish a spiroplasma culture.

2. The method of claim 1, wherein the media mixed with egg fluid is cultured for more than 5 weeks to establish a spiroplasma culture.

3. The method of claim 1, wherein the media mixed with egg fluid is cultured for more than 6 weeks to establish a spiroplasma culture.

4. The method of claim 1, wherein the mammal is a bovine, sheep or human.

5. The method of claim 4, wherein the mammal is a bovine or human.

6. The method of claim 5, wherein the mammal is a bovine.

7. The method of claim 1 wherein the mammal has been diagnosed with having Creutzfeldt-Jakob disease, scrapie or bovine spongiform encephalopathy.

8. The method of claim 1, wherein the tissue sample is neuronal tissue.

9. The method of claim 1, wherein the media is M1D, SP4, or H-I media.

10. A method of isolating and culturing spiroplasma from an animal selected from the group consisting of bovine, sheep, goat, cervid, cat, mink and ruminant which comprises: obtaining a tissue sample from a bovine; inoculating embryonated eggs with the tissue sample; mixing fluid from the inoculated egg with culture media; culturing the media mixed with the egg fluid at approximately 32° C. to establish a spiroplasma culture.

11. The method of claim 10, wherein the media mixed with the egg fluid is cultured for more than one month to establish a spiroplasma culture.

12. The method of claim 11, wherein the media mixed with egg fluid is cultured for more than 5 weeks to establish a spiroplasma culture.

13. The method of claim 11, wherein the media mixed with egg fluid is cultured for more than 6 weeks to establish a spiroplasma culture.

14. The method of claim 10, wherein the animal is a bovine.

15. The method of claim 14 wherein the bovine has been diagnosed with having bovine spongiform encephalopathy.

16. The method of claim 11, wherein the tissue sample is neuronal tissue.

17. The method of claim 11, wherein the media is M1D, SP4, or H-I media.
